(19) United States
(12) Patent Application Publication
(10) Pub. No.: US 2009/0012008 A1
(43) Pub. Date: Jan. 8, 2009

(54) Pharmaceutical Composition for Photodynamic Therapy and a Method for Treating Oncological Diseases by Using Said Composition

(76) Inventors: Vladimir Petrovich Laptev, Monino (RU); Valentin Viktorovich Petrov, Moscow (RU); Sergey Mikhailovich Andreev, Moscow (RU); Natella Georgievna Panferova, Kotelniki (RU); Valentina Semenovna Romanova, Moscow (RU); Alexandr Evgenievich Ovchinnikov, Moscow (RU)

Correspondence Address: LADAS & PARRY LLP, 26 WEST 61ST STREET, NEW YORK, NY, 10023, US

(21) Appl. No.: 12/086,284

(22) PCT Filed: Nov. 28, 2006
(86) PCT No.: PCT/RU2006/000637

§ 371 Date: Aug. 7, 2008

(30) Foreign Application Priority Data
Dec. 26, 2005 (RU) 2005140680

(51) Int. Cl. A61K 38/05; A61K 38/02; A61P 35/00; A61K 31/197

(52) U.S. Cl. 514/19; 514/2; 514/561; 977/904

(57) ABSTRACT

The present invention relates to the field of medicine, particularly to oncology and provides a pharmaceutical composition intended for the photodynamic therapy of malignant tumors, comprising a therapeutically effective amount of at least one of derivatives of fullerene C₆₀ selected from the group consisting of a compound in which a fullerene C₆₀ molecule covalently binds to one molecule of an amino acid or a dipeptide, or its pharmaceutically acceptable derivative, or a complex of a compound with biocompatible synthetic polymers or biopolymers and tetrapyrroles, or a conjugate of a compound with an amino compound. Said compounds after accumulation in cells and photoirradiation transform molecular oxygen into singlet oxygen, generate free radicals, and also trigger biological processes inhibiting the vital activity of tumor cells. The present invention also relates to a method of photodynamic therapy of malignant tumors, in which the pharmaceutical composition of the present invention is used as the photosensitizer.

### FIELD OF THE ART TO WHICH THE INVENTION RELATES

The present invention relates to medicine and is associated with the provision and application of new photosensitizers (PSs) for photodynamic therapy (PDT) of malignant tumors. PDT is a method of local activation by light of PS accumulated in a tumor, this, in the presence of tissue oxygen, leading to the development of photochemical reactions destroying tumor cells. The present invention proposes to use as the PS amino acid and peptide derivatives of fullerene C₆₀ and their complexes with biocompatible polymers and tetrapyrroles such as chlorine e₆ and their metallocomplexes. Said compounds after accumulation in cells and photoirradiation transform molecular oxygen into singlet oxygen, generate free radicals, and also trigger biological processes inhibiting the vital activity of tumor cells.

### STATE OF THE ART

It is known that upon introduction of PSs into an organism they selectively accumulate in tumor cells, and their subsequent irradiation by light of a low-power laser with a corresponding wavelength leads to the generation of free radicals and singlet oxygen, destroying the tumor. PSs can be administered intravenously, per os or applied externally to the tumor tissue. After selective accumulation of PSs in tumor cells, they are irradiated by light in a visible region, which in the presence of oxygen causes their destruction. The mechanism of action of PDT can be represented as follows: a PS molecule, having absorbed a quantum of light, passes into the excited triplet state and enters into photochemical reactions of two types. In the first type of reactions there takes place interaction directly with the molecules of a biological substrate, which finally leads to the formation of free radicals. In the second type of reactions there takes place interaction of excited PS with a molecule of oxygen with the formation of singlet oxygen which is cytotoxic for living cells because of strong oxidizing effect. The physicochemical properties of PSs should provide sufficiently high selectivity of their accumulation in the tumor tissue, low toxicity for normal cells, and high phototoxicity for malignant cells, associated with the high quantum yield of singlet oxygen, and also have good chemical stability and ability to elimination from the organism (Wyss P. History of Photomedicine II Wyss P., Tadir Y., Tromberg B. J., Haller U. (eds): Photomedicine in Gynecology and Reproduction.—Basel: Karger, 2000, p. 4-11; http://www.magicray.ru/RU/article/modern.html). The currently employed PSs are derivatives of phthalocyanines or natural tetrapyrroles (hematoporphyrin, bacteriochlorine); all of them are water-soluble at pH>7 and have characteristic absorption bands in the 400-650 nm region. At the moment commercial preparations: <<Photoditazine>> (bacteriochlorine e₆), <<Photogem>> (hematoporphyrin), “Photosens” (phthalocyanine), <<Alasens>> (5-aminolevulinic acid—precursor of protoporphyrin IX), <<Photofrin II>> (hematoporphyrin) have become most popular (Materialy IV Vserossijskoj Nauchno-prakticheskoj Konferentsii <<Otechestvennye Protivoopukholevye Preparaty>>. Moscow, Mar. 16-18, 2005, pp. 1-36; Historical aspects of the development of photodynamic therapy. http://www.magicray.ru/RU/lecture/html). One of limitations of the present-day PDT is small depth of light penetration through biological tissues because of a relatively short-wave part of the spectrum being used: 600-650 nm.

An alternative class of promising PSs, yet not employed in medicine, are compounds based on fullerenes. Fullerenes are a recently discovered new allotropic molecular form of carbon. They are spherical polyhedral molecules about 1 nanometer in diameter containing 60 and more carbon atoms. Spherical fullerene with 60 carbon atoms (C₆₀) is the cheapest and it is produced now in large-tonnage quantities. In view of its high hydrophobicity fullerene is usually modified, by introducing into the core hydrophilic groups, thereby solubility or dispersancy in aqueous media being imparted to it (Sidorov L. N., Yurovskaya M. A, et al., “Fullereny”. M.: “Ekzamen” Publishers, 2005, 688 pp.) Fullerene and its derivatives are electronegative compounds (“electronic sponge”). Therefore, in the interaction of the fullerene core with a molecule having electron-donor activity (nucleophile), a charge transport adduct is formed. Such adducts have a valuable photoactive property—the photoirradiation induces effective intramolecular electron transport from the donor molecule into the fullerene core, that leads to a sharp change of the optical properties of the adduct. In physiological conditions such changes lead to the generation of active forms of oxygen (Mizuseki H., Igarashi N., Belosludov R. V., Farajian A. A., Kawazoe Y. Theoretical Study of Chlorine-Fullerene Supramolecular Complexes for Photovoltaic Devices Jpn. J. Appl. Phys., 2003, 42/4B, 2503-2505).

Numerous experiments demonstrate that fullerene C₆₀ is an effective PS, converting oxygen into the singlet state (Yamakoshi Y, Sueyoshi S., Miyata N. Biological activity of photo-excited fullerene. Kokuritsu Iyakuhin Shokuhin Eisei Kenkyusho Hokoku, 1999, 117, 50-60; Arbogast J. W., Foote C. S. J. Am. Chem. Soc, 1991, 113, 8886-8891). The presence in fullerene derivatives of a weak absorption band in the 700 nm region makes it possible to use C₆₀ compounds for the PDT owing to effective penetration of light with such wavelength through biological tissues. C₆₉ derivatives accumulate in the tumor tissue due to their enhanced permeability and relative immaturity of the lymphatic system. Besides, these compounds aggregate in the aqueous medium due to the association of the hydrophobic cores of C₆₀, and an increase of the molecular mass promotes their more intensive absorption by tumor cells. C₆₀ compounds are also capable to produce free radicals, especially in deficiency of oxygen (Elisa M. M. , Gabriela A. M. et al. Porphyrin-fullerene C60 dyads with high ability to form photoinduced charge-separated state as novel sensitizers for photodynamic therapy. Photochem. PhotobioL, 2005, 81/4, 891-897; Cardullo F., Isaacs I., Diederich F., Gisselbecht J.-P., Boudon C, Gronn M., Chem. Commun., 1996, 797-799). Local photoirradiation of mice with transplanted fibroid tumor, pretreated with fullerene derivatives, led to the tumor necrosis without damaging healthy tissues (Tabata Y., Murakami Y, Ikada Y. Photodynamic effect of polyethylene glycol-modified fullerene on tumor, Jpn. J. Cancer Res., 1997.88, 1108-1116; Tabata Y, Murakami Y, Ikada Y. Antitumor effect of poly(ethylene glycol)-modified fullerene. Fullerene Sci. Technol., 1997, 5, 989-1007). A conjugate of fullerene C₆₀ with polyethylene glycol (PEG) caused active necrosis of the transplanted tumor in mice in a dose of 0.4 mg/kg bodyweight, the healthy tissue surrounding the tumor being not affected in any manner. Cancer cells selectively accumulated C₆₀-PEG after its intravenous administration—in comparison with normal skin and muscles the selectivity factor for tumor was 2.5 and 17, respectively (Tabata Y., Ikada Y. Biological function of fullerene. Pure Appl. Chem., 1999, 71 (11), 2047-2059). In experiments in vitro with tumor cells of the HeLa S3 line the cytotoxic activity of fullerocyclopentane was manifest only upon irradiation with light (Schwenninger R., Muller, Krautler J. Am. Chem. Soc, 1997, 119, 9317).

Investigations into the toxicity and pharmacokinetics of fullerenes containing hydrophilic addends have shown that these substances are eliminated from the organism and have low acute toxicity (Wilson L. I, Cagle D. W., Thrash., Kennel S. I, Mirzadeh S., Alford J. M., Ehrhardt G. J. Metallofullerene drag design. Coordination Chem. Rev., 1999, 190-192, 199-207; Nelson M. A., Domann F. E., Bowden G., Hooser S., Fernando Q., Carter D. E. Effects of acute and subchronic exposure of topically applied fullerene extracts on the mouse skin. Toxicol. Ind. Health, 1993, 9, 623-630), i.e. in dozes usually employed for the PDT they are safe. Thus, non-modified fullerene C₆₀ produces no effect on the viability and proliferation rate of fibroblasts and keratinocytes (W. A. Scrivens, Tour J. M., Creek K. E., Pirisi L. Synthesis of ¹⁴C-labeled C60, its suspension in water, and its uptake by human keratinocytes // J. Am. Chem. Soc. 1994.116. P. 4517-518). These data correlate well with the results of experiments on the cytotoxicity of C₆₀ on animal models and cultures of dendritic human cells (Rancan F., Rosan S., Boehm F., Cantrell A., Brellreich M., Schoenberger H., Hirsch A., Moussa F. Cytotoxicity and photocytotoxicity of a dendritic C(60) mono-adduct and a malonic acid C(60) tris-adduct on Jurkat cells. J. Photochem. Photobiol., 2002, 67, 157-162; Lin A. M., Fang S.-F., Lin S.-Z., Chou C.-K., Luh T.-Y., Hoa L.-T. Local carboxyfullerene protects cortical infarction in rat brain. Neurosci. Res., 2002, 43, 317-321).

### DISCLOSURE OF THE INVENTION

The present invention relates to medicine and is directed to the development of new pharmaceutical compositions for photodynamic therapy of tumoral diseases by introducing PSs based on water-soluble amino acid and dipeptide derivatives of fullerene C₆₀ and their non-covalent complexes or covalent conjugates and methods of photodynamic therapy of tumoral diseases, contemplating administration of pharmaceutical compositions of the present invention.

Therefore, in its first aspect the present invention is concerned with a pharmaceutical composition for photodynamic therapy of malignant tumors, containing a therapeutically effective amount of at least one of fullerene C₆₀ derivatives selected from the group consisting of:
a) a compound in which the fullerene C₆₀ molecule covalently binds to one molecule of an amino acid, or its pharmaceutically acceptable salt, or pharmaceutically acceptable derivative;
b) a compound in which the fullerene C₆₀ molecule covalently binds to one molecule of a dipeptide, or its pharmaceutically acceptable salt, or a pharmaceutically acceptable derivative;
c) a non-covalent complex of the compound characterized above in sub-items a) or b) or its pharmaceutically acceptable salt, or a pharmaceutically acceptable derivative with a photosensitizer from the class of tetrapyrroles, or a photosensitizer from the chlorine-e₆ series or a metallocomplex of chlorine-e₆, or a synthetic polymer, or a biopolymer; and
d) a conjugate of the compound characterized above in sub-items a) or b), with an amino compound;
   and a pharmaceutically acceptable excipient or solvent.

In one of the preferred embodiments of the inventive pharmaceutical composition the amino acid, covalently bonded to the fullerene C₆₀ molecule is an L- or D-amino acid selected from the group consisting of (international three-letter code): Ala, Arg, Asn, Asp, Cys, Gly, Glu, Gin, His, Ile, Leu, Lys, Met, Phe, Pro, Ser, Thr, Trp, Tyr, Val, γ-aminobutyric acid (Aba), ε-aminocaproic acid (Acp), citrulline, ornithine.

In one more embodiment of the inventive composition the dipeptide covalently bonded to the fullerene C₆₀ is a dipeptide comprising two L- or D-amino acids independently selected from the group consisting of (international three-letter code): Ala, Arg, Asn, Asp, Cys, Gly, Glu, Gln, His, Ile, Leu, Lys, Met, Phe, Pro, Ser, Thr, Trp, Tyr, Val, γ-aminobutyric acid (Aba), ε-aminocaproic acid (Acp), citrulline, ornithine.

In its next aspect the invention concerns a method of photodynamic therapy of a human oncological disease, characterized in that a patient in need of such treatment is administered as a photosensitizer the pharmaceutical composition related to the first aspect of the invention.

In one of the embodiments of the method of the present invention the oncological disease is selected from the group consisting of carcinomas, sarcomas, lymphomas, leukaemias, blastomas.

In another preferred embodiment of the method the pharmaceutical composition is administered intramuscularly, intraperitoneally, topically, orally, intravenously, intratumorally, intranasally.

In still another preferred embodiment of the method of the present invention the pharmaceutical composition is administered in a daily dose amounting from 0.5 to 50 mg/kg body weight.

The effect of the treatment is achieved by the photoactivation of PSs accumulated in the tumor, for which purpose use is made of laser and light-emitting diode radiation in a 640-680 nm region. A combination of an amino acid or dipeptide fullerene derivative with a hydrophilic electron donor (tetrapyrrole compounds being such), makes it possible to broaden the irradiation spectrum to 1 μm (near IR-region) and thereby to increase the depth of light penetration into the tissue. Tetrapyrroles and, in particular, chlorine-e₆ are good electron donors having a developed mobile conjugated π-electron system with a high degree of selectivity of accumulation in malignant cells. Therefore these compounds are good partners for preparing complexes with fullerene. For example, such pair in a physiological medium upon irradiation displays photodynamic effect with respect to hepatocytes, producing singlet oxygen in the presence of oxygen or free radicals in the deficiency of oxygen (Elisa M. M., Gabriela AND. M, Silber R. V., Durantini J. I, Edgardo N. Porphyrin-fullerene C60 Dyads with High Ability to Form Photoinduced Charge-separated State as Novel Sensitizers for Photodynamic Therapy. Photochem. Photobiol., 2005, 81, 891-897).

An advantage and a new approach consist in reciprocal complementation in the absorption of photoradiation: in bacteriochlorine (chlorine-e₆), whose absorption spectrum is presented in FIG. 1 is 635 nm and 420 nm, while in fullerene derivatives it is up to 1 μm, catching the near-IR region (FIG. 2), this enhancing the photochemical activity of the PDT. Photoirradiation in the near-IR region provides light penetration to a greater depth, and this allows treating a wider range of oncological diseases, for example, tumors of internal organs.

Another advantage consists in that fullerenes are carbon hydrophobic clusters. This, on the one hand, promotes their greater affinity for cell membranes (Andreev I. M. Romanova V. S. Petrukhina A., S. M. Andreev. Amino acid derivatives of fullerene C60 behave as lipophilic ions penetrating through biomembranes. Physics of the Solid State, 2002, 44 (4), 683-685; Kotelnikova R. A., Bogdanov G. N., Frog E. C, Kotelnikov A. I., Romanova V. S., Andreev S. M, Kushch A. A, Fedorova N. E., Medzhidova A. A., G. G. Miller. Nanobionics of pharmacologically active derivatives of fullerene C₆₀. J. Nanoparticle Res., 2003, 5, 561-566), and, on the other hand, this stimulates absorption of such compounds by tumor cells, since because of aggregation their molecular mass increases (Seymour L. W. Passive tumor targeting of soluble macromolecules and drug conjugates. Crit. Rev. Ther. Drug Carrier Syst., 1992, 9, 135-187; Andreev S. M., Petrukhina A. O., Romanova V. S., Babakhin A. A., Petrov R. V., Immunogenic and allergenic properties of fullerene conjugates with amino acids and protein. Dokl. RAN, 2000, 370, 261-264). The large mass also promotes longer retention of the substance in tumor cells.

Complexation of biocompatible polymers and charged tetrapyrrole compounds with amino acid and peptide fullerene derivatives sharply increases the solubility of the latter, making it possible not to limit the doses of the pharmacologically active compound being administered.

Other aspects of the present invention will be obvious from the accompanying drawings, the detailed description and the set of claims.

### LIST OF FIGURES

For a clearer understanding of the claimed invention, and also for demonstrating its specific features and advantages, given below is a detailed description of the invention with references to the figures of the drawings, in which

FIG. 1 demonstrates absorption spectrum of chlorine e₆ (X-axis: nm).

FIG. 2 demonstrates absorption spectrum of C₆₀-Aba.

FIG. 3 demonstrates absorption spectrum of C₆₀-Arg-Arg complex with chlorine e₆.

FIG. 4 demonstrates absorption spectrum of C₆₀-Aba complex with polyvinyl pyrrolidone.

FIG. 5 demonstrates IR absorption spectrum of C₆₀-Aba-Phe-NH₂.

FIG. 6 shows photoactivation by laser and light-emitting diode radiation.

FIG. 7 shows osmolar death of tumor cells after PDT.

FIG. 8 shows adhesion and death of cells in ascites after PDT.

FIG. 9 shows membrane-contact interactions of immunocompetent cells in ascites.

FIG. 10 demonstrates intramuscularly transplanted embryocarcinoma.

FIG. 11 Demonstrates tumor necrosis on the 5^{th} day after irradiation.

FIG. 12 Demonstrates tumor necrosis 2 weeks after irradiation.

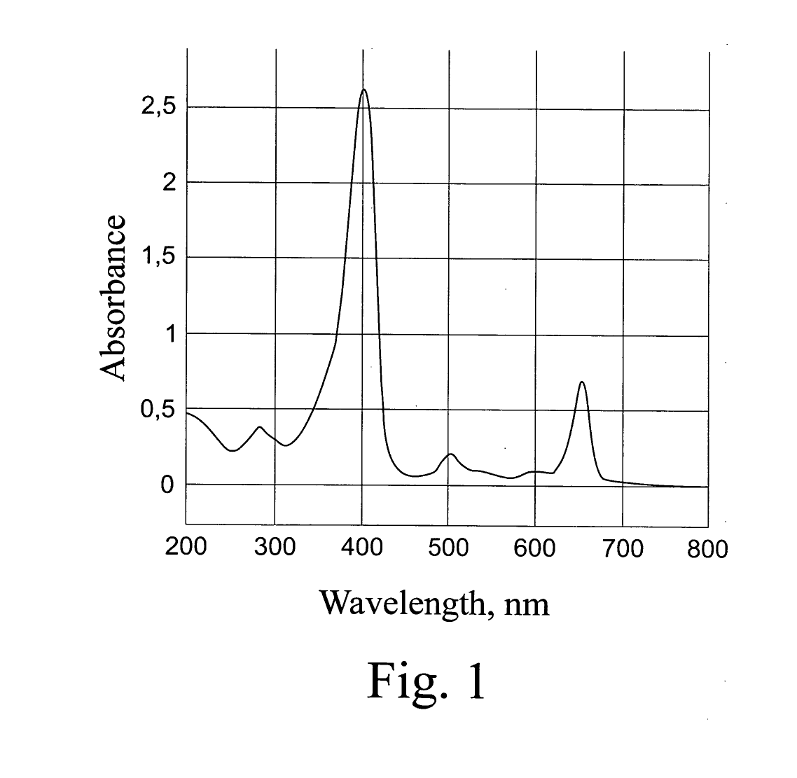

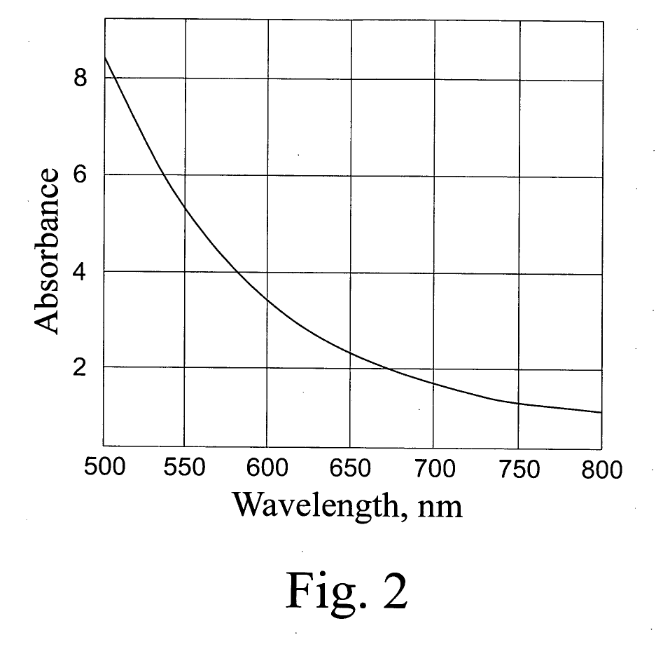

### REALIZATION OF THE INVENTION

The range of water-soluble fullerene derivatives employed within the framework of the present invention, but not limited only to these derivatives, comprises C₆₀ adducts with amino acids of L- or D-series or dipeptides formed by amino acids of L- or D-series. Preferable for the purposes of the present invention are such amino acid derivatives of fullerene C₆₀, in which the amino acid covalently bonded to the fullerene molecule is an L- or D-amino acid selected from the group consisting of (international three-letter code): Ala, Arg, Asn, Asp, Cys, Gly, Glu, Gin, His, Ile, Leu, Lys, Met, Phe, Pro, Ser, Thr, Trp, Tyr, Val, γ-aminobutyric acid (Aba), ε-aminocaproic acid (Acp), citrulline, ornithine. Preferable for the purposes of the present invention are such dipeptide derivatives of C₆₀ fullerene, in which the dipeptide covalently bonded to fullerene, is dipeptide, consisting of a combination of two L- or D-amino acids independently selected from group consisting of (international three-letter code): Ala, Arg, Asn, Asp, Cys, Gly, Glu, Gin, His, Ile, Leu, Lys, Met, Phe, Pro, Ser, Thr, Trp, Tyr, Val, γ-aminobutyric acid (Aba), ε-aminocaproic acid (Acp), citrulline, ornithine.

Carboxyl group of the amino acid or dipeptide bonded to the fullerene molecule can be free, and also be in the form of a pharmaceutically acceptable salt or in the form of physiologically acceptable derivatives, such as amide, ester, alkylamide, arylamide, or hydroxyalkyl(aryl)amide. The scope of the invention also comprises other physiologically acceptable derivatives of amino acid or dipeptide derivatives of fullerene, in particular such, in which other functional groups which are present in the formulation of amino acids are modified, in particular, hydroxyl, guanidine, amino, thiol groups. Methods of modifying such functional groups are well known to those skilled in the art. It is implied that upon introducing such physiologically acceptable derivatives regeneration of the active component in vivo will occur.

The term “pharmaceutically acceptable salt” should be understood as denoting such salt which is acceptable from the pharmaceutical point of view and which has the desirable pharmacological activity of the initial substance. In the context of the present invention pharmaceutically acceptable salts can be formed at the free C-terminal carboxyl group of the amino acid or peptide. Such salts comprise basic addition salts formed by inorganic or organic bases. To inorganic bases acceptable in the aboveindicated sense there belong, in particular, ammonium hydroxide, hydroxides of alkali metals such as potassium, lithium, etc., hydroxides of alkaline-earth metals such as calcium, magnesium, etc., aluminum hydroxide and the like; to the organic bases there belong, for example, ethanolamine, diethanolamine, triethanolamine, N-methylglucamine, and the like.

In the case when the formulation of an amino acid or peptide fullerene derivative comprises an amino acid having additional functional groups capable of forming salts, besides an α-amino group and a carboxyl group, as, for example, one more carboxyl group (Asp, Glu), or an amino group (Lys), or a guanidine group (Arg), or an imidazole group (His), in addition to basic addition salts acid-addition salts can be obtained, formed by inorganic or organic acids. To the inorganic salts, pharmaceutically acceptable in the aboveindicated sense, there belong, in particular, hydrochloric, hydrobromic, sulfuric, nitric, phosphoric acids, and the like; to the organic acids there belong, for example, acetic, propionic, pyroracemic, lactic, citric, cinnamic, succinic, malonic, maleic, fumaric, benzoic, ethanesulfonic, methanesulfonic, gluconic, trimethylacetic, salicylic, stearic, muconic acid, and the like. An exhaustive information about pharmaceutically acceptable salts can be found in a comprehensive guide Remington's Pharmaceutical Sciences, 18^{th} edition, A. R. Gennaro, Ed., Mack Publishing Company, 1990 which is included in the present description in all completeness by reference.

The indicated compounds are prepared by the known methods by direct addition of amino acids or dipeptides to the fullerene core (Parnes Z. N., Romanova V. S., Andreev S. M., Petrukhina A. O, Vol'pin M. E., “Adjuvants”. Letters patent No. 2129436, 1997; Romanova V. S., Tsyryapkin V. A., Lyakhovetsky Yu. A., Pasrnes Z. N., Vol'pin M. E., Izv. RAN, ser. Khim., 1994, 1151). It should be noted that the given class of compounds is physiologic, since for their preparation nontoxic amino acids, dipeptides and other biocompatible compounds are used, and their low toxicity has been confirmed by a number of biological tests (Andreev S. M., Petrukhina A. O., Romanova V. S., Babakhin A. A, Petrov R. V. Immunogenic and allergenic properties of fullerene conjugates with amino acids and protein. Dokl. RAN, 2000, 370, 261-264; Miller G. G., Romanova V. S., Pokidysheva. et al. Inhibition of HIV reproduction with the help of amino acid and dipeptide derivatives of fullerene C60. Antibiotiki I Khimioterapiya, 2004, 49 (12), 3-12; Medzhidova M. G., Abdullaeva M. V, Fedorova N. E, Romanova V. S., Kushch A. A. Antiviral activity of amino acid derivatives of fullerene in citomegaloviral infection in vitro. Antibiotiki I Khimiotterapiya, 2004, 49 (8-9), 13-20). It has been shown, that a sufficient therapeutic dose of the preparation for obtaining PDT-effect in tumors and minimal release of the excess of the administered preparation through the liver into the intestine is 7-15 mg/kg bodyweight, this being by an order of magnitude lower than the administered subtoxic and toxic doses.

Noncovalent complexes of amino acid and dipeptide derivatives of fullerene C₆₀ with tetrapyrrole compounds, synthetic polymers and biopolymers are prepared by mixing solutions of the components to be complexed in a suitable medium (water, physiologic solution, Hanks' solution, etc.) at pH, allowing to transform C₆₀ derivatives into soluble form, and keeping the resulting mixture for a definite period of time at temperatures of from about 0 to about 30° C. The target product is isolated by dialysis or chromatographically with subsequent lyophilization of the dialyzate or the required fraction. Since fullerene derivatives are carboxy-containing compounds, their covalent conjugation is carried out by condensation with amine-containing biodegradable compounds, e.g. such as peptides and proteins (gelatin, albumins). For the preparation of complexes use is made of biocompatible polymers, from the category of blood substituents, such as, for example, low-molecular synthetic polyvinyl pyrrolidone, polyvinyl alcohol, polyglukin, dextran, polyethylene glycol, hydroxyethyl starch, polymethyl methacrylates, natural—gelatinolum , and also other medical-purpose nontoxic polymers.

Covalent complexes of amino acid C₆₀ derivatives with amino-containing compounds are prepared with the use of suitable condensing agents, for example, trifluoroacetoxy succinimide (Andreev S. M., Sidorova M. V., Rakova O. A., Tsvetkov D. E., Fonina L. A. Synthesis of N-oxysuccinimide esters of organic acids and carboxyl-containing polymers with the use of N-trifluoroacetoxy succinimide. Bioorg. Khimiya, 1987, 13 (5), 696-700) or carbodiimides, for example, dicyclohexyl carbodiimide (Sheehan J. C., Hess G. P. J Am. Chem. Soc, 1955, 77, 1067).

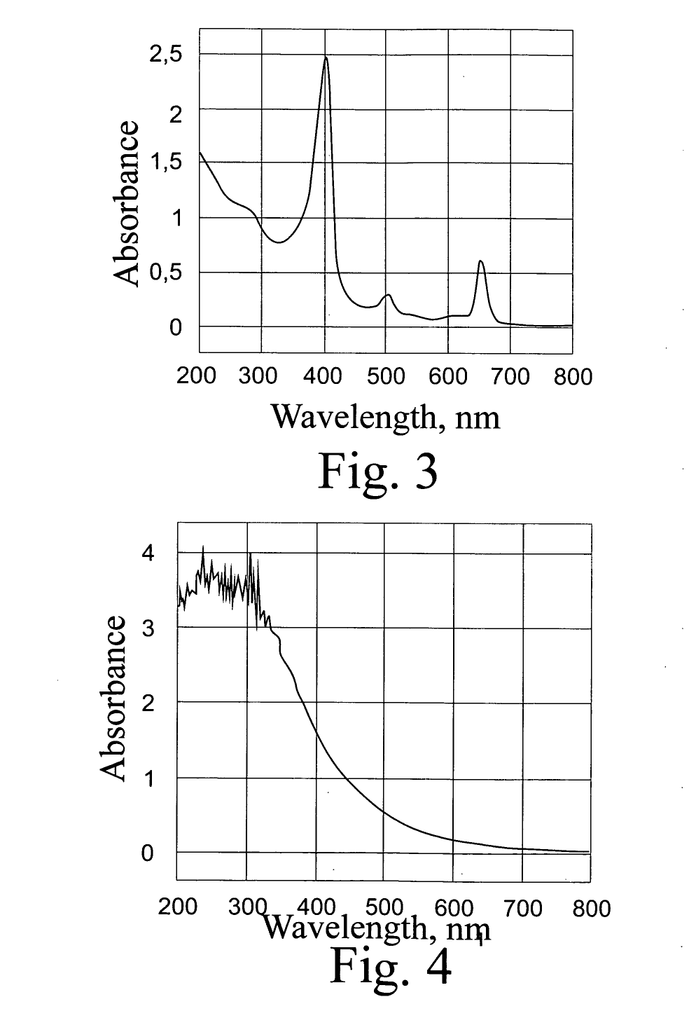

Spectrophotometric analysis of fullerene complexes containing chlorine e₆ testifies to the preservation of absorption bands (FIG. 3), characteristic of this compound. Quenching of e₆ fluorescence does not occur.

An analysis of acute toxicity of fullerene derivatives has shown that in doses below 70 mg/kg these compounds are low-toxic. It is necessary to note that for photoactive compounds (hematoporphyrins, chlorines) there exists individual sensitivity to preparations, even in an weight- sex- and age-identical syngenic system of animals carrying out photodynamic therapy of tumors. The photochemical effect on malignant cells after laser irradiation can vary within significant limits. Probably, fullerene compounds also are not an exception in this series of photosensitizers. Our experiments in studying the action of amino acid derivatives of fullerene on human and murine erythrocytes have shown that even in high concentrations (400 mM) these compounds do not cause hemolysis or agglutination, cells during incubation do not undergo morphological changes. Parenteral administration of comparatively high doses of C₆₀-Ser (80 mg/kg) to mice did not produce any effect on the behavior and viability of mice within 6 months, being indicative of low toxicity of amino acid fullerene derivatives (Andreev S. M., Petrukhina A. O., Romanova V. S., Babakhin A. A., Petrov R. V. Immunogenic and allergenic properties of fullerene conjugates with amino acids and protein. Dokl. RAN, 2000, 370, 261-264).

Hence, the inventive pharmaceutical composition is a combination of low-toxic components: a covalent fullerene-amino acid/dipeptide adduct, biocompatible polymers and tetrapyrrole compounds (for example, chlorine e₆ or its metallocomplexes).

The inventive compositions suitable for application in the photodynamic therapy humans can be prepared in accordance with methods known to a specialist skilled in the art, particularly by mixing with pharmaceutically acceptable excipients.

In the context of the present description the term “pharmaceutically acceptable” should be understood as denoting such excipient as a carrier, solvent or any other target additive which do not cause undesirable side effects in an individual to which they are administered and do not interfere with the realization of the target biological function upon introduction of PSs in the organism of the individual. Such pharmaceutically and physiologically acceptable means are well known to those skilled in the art (see, for example, Remington's Pharmaceutical Sciences, 18^{th} edition, A. R. Gennaro, Ed., Mack Publishing Company—1990; Handbook of Pharmaceutical Excipients, 3^{rd} edition, A. Kibbe, Ed., Pharmaceutical Press—2000). Such excipients can be buffers, dispersants, preservatives, stabilizers, carriers, fillers, solvents, etc.

In particular, to pharmaceutically acceptable excipients there belong pharmaceutically acceptable salts which promote maintenance of pH, isotonicity, and also impart other useful properties to the pharmaceutical composition. For example, inorganic salts of sodium, potassium, lithium, ammonium, and also organic salts of primary, secondary or tertiary amines, and of amino acids, belong to such salts. Various organic salts, such as salts of acetic acid, propionic acid, pyruvic acid, maleic acid and of other acceptable acids can be prepared and used for the purposes of present invention as well.

For maintaining osmotic pressure, the pharmaceutical compositions of the present invention can comprise such tonicity regulating agents as sucrose, glucose, sodium chloride, and also polyhydric sugar alcohols such as glycerol, erythritol, arabitol, xylitol, sorbitol or mannitol.

For maintaining physiological pH values, the pharmaceutical compositions of the present invention can comprise physiologically acceptable buffer agents, such as, for example, Hepes, phosphate, citrate, succinate, tartrate, fumarate, gluconate, oxalate, lactate, acetate or histidine buffers and combinations thereof.

In the case when the pharmaceutical composition of the present invention is intended for topical administration, it can be in the form of ointment, liniment, emulsion, suspension, lotion, etc. As their base substances known to those skilled in the art can be used, e.g. oils or fats of synthetic and animal or vegetable origin, such as vaseline, petrolatum, lanolin, soybean oil, corn oil, sunflower oil, coconut oil, pork fat, glycerin, fatty acids, polyhydric alcohols and their esters with fatty acids, transdermal transport enhancers, and other similar pharmaceutically acceptable components.

The pharmaceutical composition of the present invention comprises effective amount of an active principle—photosensitizer. In a context of the present Application “effective amount” should be understood as such amount of the inventive PSs, which is sufficient for producing the desired effect on the condition in connection with which they are administered to an individual. A precise amount will depend on particular circumstances and can be assessed by a person skilled in the art with the aid of known techniques. Usually, the amount should be such as to produce the desired therapeutic action (necrosis, inhibition) on the malignant tumor in a human to which it is administered. To one skilled in the art it will be clear that the effective amount will depend, inter alia, on the type of tumor, on the mode of administration, on the formulation of the composition, on whether the composition is administered singly or in combination with other medicaments, on the general well-being of the individual, on the reactivity of his/her organism. The compositions of the present invention will be usually administered in the doses within a range of from about 0.5 mg to about 50 mg.

Since the pharmaceutical compositions of the present invention can be administered intramuscularly, intraperitoneally, topically, orally, intravenously, intratumorally, intranasally, it is clear to a person skilled in the art that the compositions should be predominantly in sterile form. For providing sterility, various known methods known in the state of the art can be used, such as filtering through a sterilizing filter with the pore size of 0.22 μm. For precluding bacterial infection, the formulation of the pharmaceutical compositions can further (optionally) comprise preservatives, for example, thimerosal, phenol.

Further the invention will be illustrated by examples, but it should be understood that examples are given exclusively with a view to providing better understanding of the essence of the claimed invention and are not intended for limiting thereof.

### EXAMPLES

### Example 1

### Preparation Of C60-Arg-Arg Complex With Chlorine e6

Dowex 1×2 resin (C1-φopMa) was transferred into OH-form by washing with 2M NaOH solution and then with water to neutral reaction. A solution of. 52 mg of C60-Arg-Arg in 2 ml of water was treated with the OH-form of Dowex 1×2 for 15 minutes, and then the filtrate was mixed with 7 mg of chlorine e6 (2 ml) and 10 μl of concentrated NH₄OH. The resulting solution was kept for 1 hour, subjected to defiltration on an Amicon PM30 membrane and lyophilized. The yield of a brown-colored powder was 46 mg. The absorption spectrum of the prepared complex is presented in FIG. 3.

### Example 2

### Preparation Of C60-Aba Complex With Polyvinyl Pyrrolidone

48.3 mg of C60-Aba Na-salt were dissolved in 7 ml of water and 3 ml of a polyvinyl pyrroilidone solution (202 mg, MW-10000) were added thereto. The solution was adjusted to pH 8, kept for 40 hours, subjected to diafiltration on an Amicon PM30 membrane, and the remaining solution was lyophilized. The yield of a light-brown powder was 190 mg. The spectrum of the prepared complex is presented in FIG. 4.

### Example 3

### Preparation Of Phenylalanine Amide (Phe-NH₂) Conjugate With C60-Aba

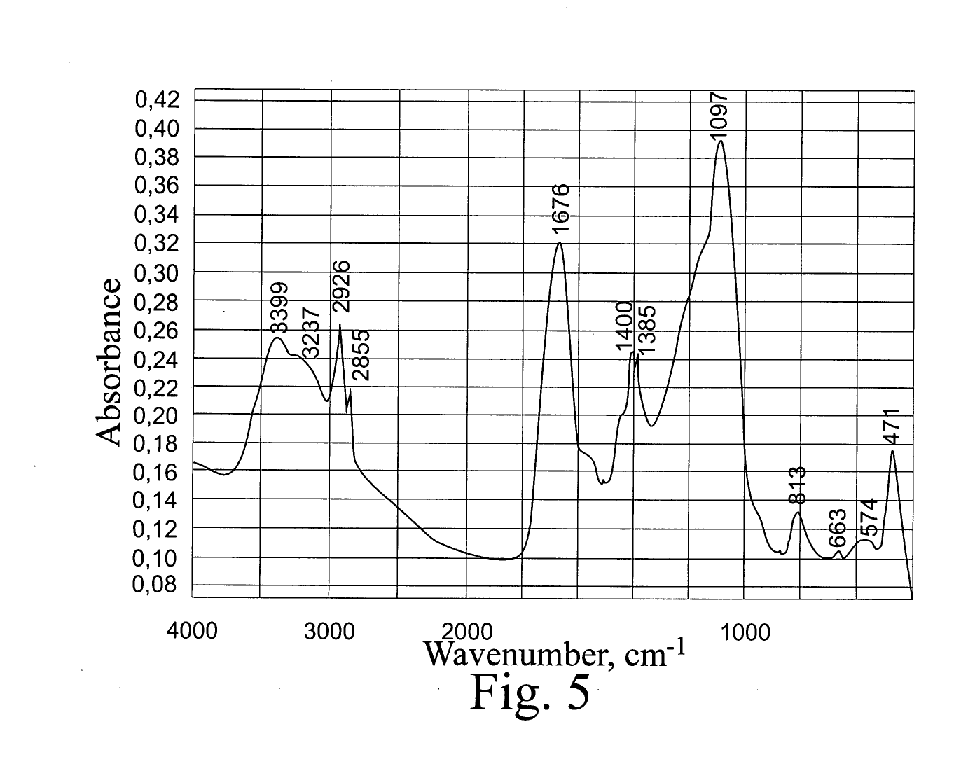

30 mg of N-oxysuccinamide and 100 μl of trifluoroacetic anhydride were mixed, kept for 20 minutes, and the solution was evaporated till white crystals of trifluoroacetoxysuccinimide (TFAS) were formed. Separately 50 mg of C60-Aba were dissolved in 1 ml of pyridine and the solution was added to the TFAS crystals the mixture was kept for 20 minutes and then 5 μl of distilled water were added. 5 mg of Phe-NH₂, were introduced into the reaction mixture. And the resulting solution was kept for 30 minutes. The reaction product was precipitated with ethyl acetate, then dissolved in 2 ml of DMFA, inverted-phase resin RP-18 was added, and kept for 30 minutes. The resin was removed by filtration, the filtrate was concentrated in vacuo, and the residue was diluted with ethyl acetate. The resulting precipitate was washed with ethyl acetate, sulfuric ether, filtered, and dried. The yield of a cream-colored powder was 43 mg. For proving the presence of a chemical bond between C60-Aba and phenylalanine amide, an amino acid analysis of the obtained product after its hydrolysis with 6N HCl for 2 hours at 160° C. was carried out. Thin-layer chromatography of the hydrolysate (system propanol:ammonia=3:1 system, development with ninhydrin) showed the presence of two amino acids, Aba and Phe. The IR-spectrum also shows the presence of strong absorption bands of NH and C═O groups (1676, 1440, 1385, 3200-3400 cm⁻¹) (FIG. 5).

### Example 4

### Preparation Of C₆₀-Acp Conjugate With Gelatinolum (10 KDa)

A mixture of crystalline TFAS prepared from 5 mg (43 μmoles) of N-oxysuccinimide and 5 μl trifluroacetic anhydride and 10 mg (11.8 μmoles) of C₆₀-Acp was dissolved in 500 μl of a DMF/pyridine (1:1) mixture, kept for 30 minutes at room temperature, and 5 μl of distilled water was added. Then the obtained solution was mixed with 30 mg medicinal gelatinolum in 1 ml of a 0.1 M phosphate-saline buffer (FSB) with pH=8.0. The reaction mixture was intensively stirred for 4 hours at room temperature, and then it was diluted to 8 ml with distilled water and dialyzed against water, using a dialysis membrane Servapore (Serva). The obtained light-brown turbid solution was centrifugated at 2000 rpm, and the supernatant was lyophilically dried. The yield of C₆₀-Acp-gelatin in the form of colored powder was 26.5 mg, the UV-spectrum: λᵣₐₓ=:268 nm.

### Example 5

### Investigations Of Acute Toxicity

For determining acute toxicity parameters, C₆₀-Aba preparation was administered intraperitoneally in the form of sterilized solution in a 0.05 M phosphate buffer with pH=7.5 intraperitoneally in single doses from 30 to 120 mg/kg bodyweight with step of 30 mg/kg bodyweight. The loss of cattle, corresponding to average LD parameters, was observed at concentrations higher than 70-80 mg/kg bodyweight. In some animals immediately after the administration of the preparation symptoms of acute toxicity were observed, expressed in adynamia, ruffling of hair and absence of protective reflex. These reactions lasted for 3-5 hours, and then the animals returned to the normal state. Further follow-up of the survived animals was continued during the 1^{st} month.

Part of the animals from the control and experimental group with toxic manifestations were used for histologic investigation 1 week after administering the preparation (liver, kidney, spleen, lungs, brain, bone marrow have been taken). No pronounced cytotoxic reactions have been detected in the above-listed organs, capable of causing death of the experimental animals.

### Example 6

### Photodynamic Therapy

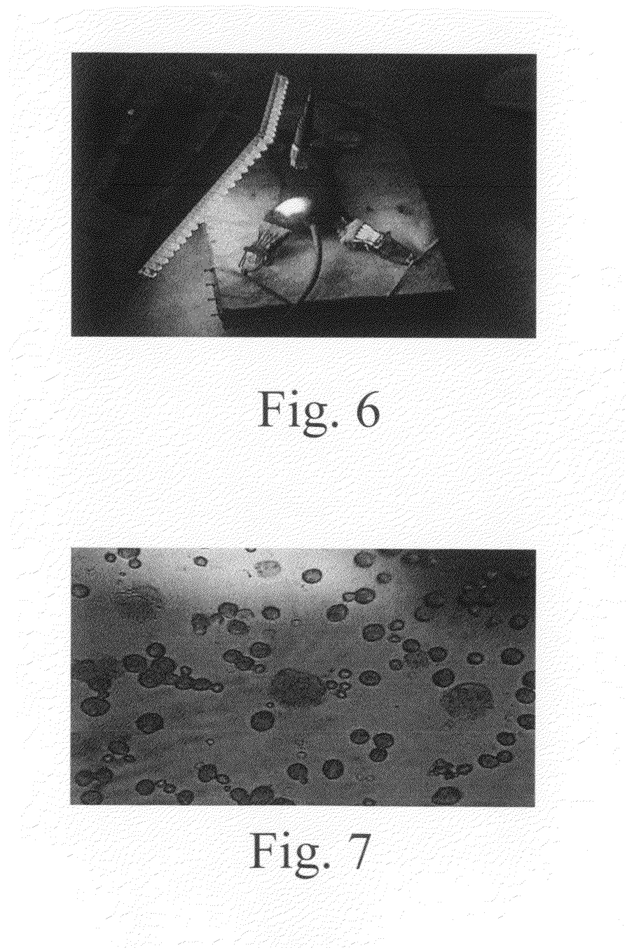

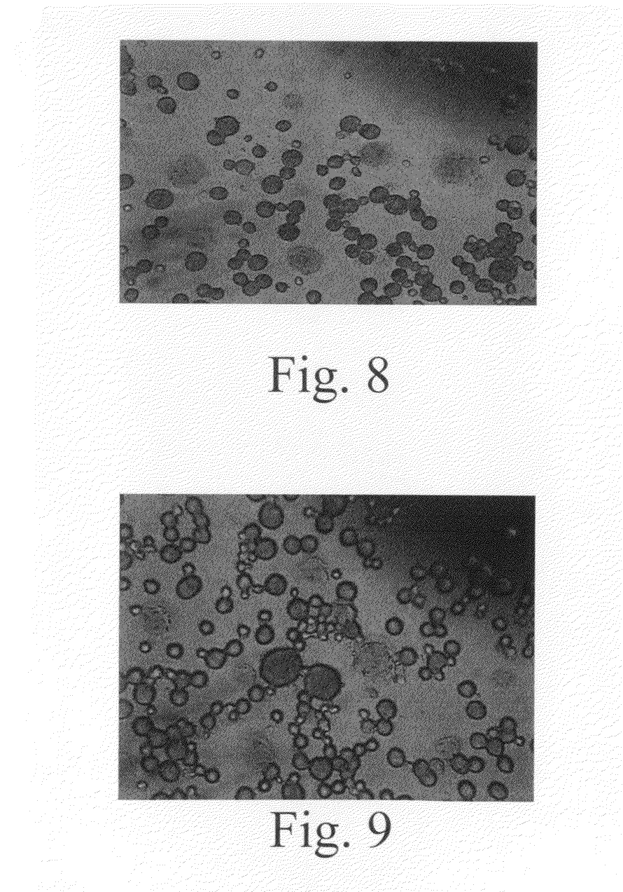

The photodynamic activity of the pharmaceutical compositions was assessed on hybrid mice hybrids F₁(CBA+C₅₇|B₆), females 20-21 grams bodyweight, with intraperitoneally transplanted embryocarcinoma (ascitic and solid growth). On the 5^{th} day of the tumor growth a composition consisting of C₆₀-Arg-Arg complex and zinc complex of chlorine-e₆ was administered intraperitoneally in a total dose of 30 mg/kg bodyweight. 2-4 hours after the administration of the composition the animals were exposed to laser radiation (662 nm and 890 nm ) in a total dose of 100 J/cm² (FIG. 6). Material for cytologic investigations was sampled 1, 24, and 48 hours after the irradiation. The results of the experiment were assessed by life expectancy in groups and by the character of morphological changes in tumor cells (see FIGS. 7-9). In the cytologic investigation of cytosmears in the experimental group a substantial increase of the number of immunocompetent cells, histiocitic elements and peritoneal macrophages has been noted. Phenomena of adhesion of immunocompetent and tumor cells with subsequent osmosis processes leading to death of tumor cells are observed. These immune reactions can be regarded as nonspecific antineoplastic activity of immunocompetent cells, provoked by photochemical processes in tumor tissue (formation of singlet oxygen and triggering of lipid peroxidation reactions). It is necessary to note polymorphism of immune cells and their membrane interaction between different types of cells.

### Example 7

### Photodynamic Therapy

PTD experiments have been carried out on Balb/c line female mice with 21-22 g bodyweight. A strain of lymphogenically metastazing embryocarcinoma (RAMN, Blokhin Russian Cancer Research Center) served as the model of tumor growth.

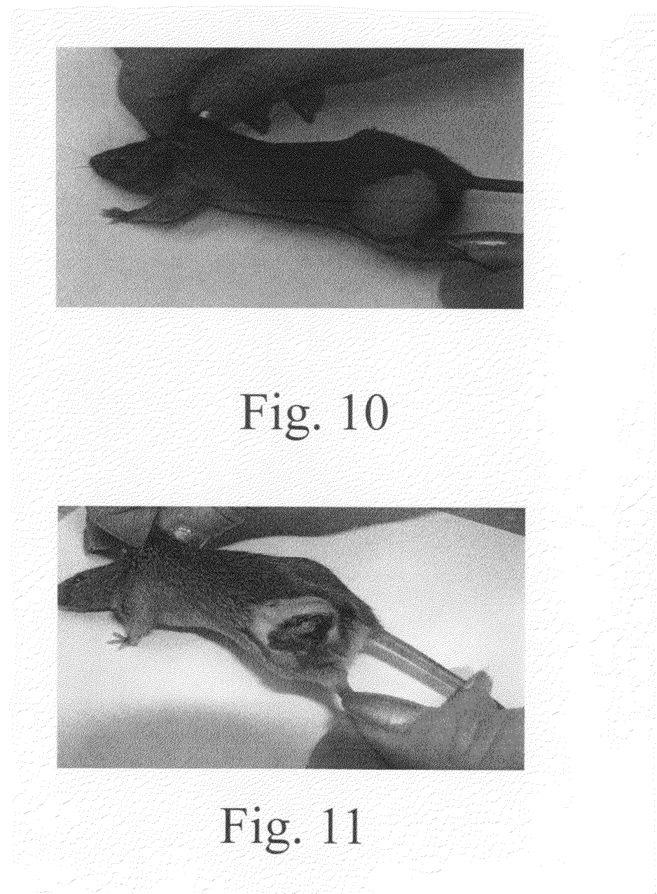

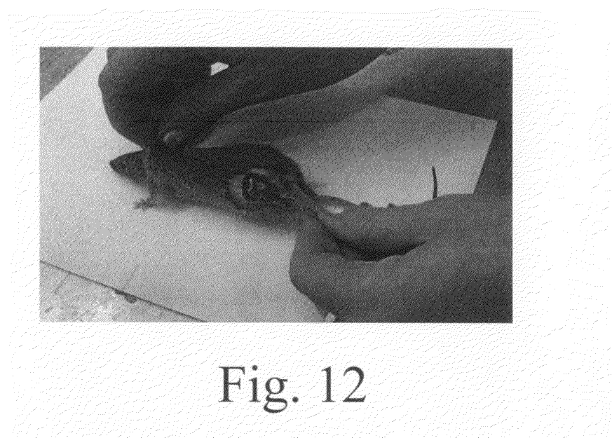

When studying photodynamic reaction on solid intramuscular tumors with intraperitoneal administration of C₆₀-Arg-Arg in physiological 0.9% solution of NaCl in a dose of 10 mg/kg and zinc complex of chlorine-e6 in a dose of 5 mg/kg bodyweight, with subsequent exposure to laser radiation in visible 615-680 nm and infrared region to 1 μm, the development of true photochemical reaction in a tumor tissue was observed not immediately during the first hours after the irradiation. The color of the tumor node changed from grey to grey-dark blue, this being indicative of the absence of thermal reaction under the action of laser radiation. Formation of a photochemical scab occurred during next 3-5 days. The depth of necrosis in the tumor node after a single PDT-effect reached 7-9 mm, this being deeper than in PDT with the use of chlorines. The dynamics of necrosis development in a tumor on a macroscopic level is shown in FIGS. 10-12.

Thus, though separate most preferable embodiments of the invention have been disclosed above in detail, a person skilled in the art will be able to make numerous additions and changes, without going beyond the framework of the present invention, which are defined by the accompanying set of claims.

1. A pharmaceutical composition for photodynamic therapy of the malignant tumors, comprising a therapeutically effective amount of at least one of derivatives of fullerene C₆₀, selected from the group consisting of:
a) a compound in which a fullerene C₆₀ molecule covalently binds to one molecule of an amino acid, or its pharmaceutically acceptable salt, or a pharmaceutically acceptable derivative;
b) a compound in which a fullerene C₆₀ molecule covalently binds to one molecule of a dipeptide, or its pharmaceutically acceptable salt, or a pharmaceutically acceptable derivative;
c) a non-covalent complex of the compound characterized above in sub-items a) or b) or its pharmaceutically acceptable salt, or a pharmaceutically acceptable derivative with a photosensitizer from the class of tetrapyrroles, or a photosensitizer from the chlorine-e₆ series or a metallocomplex of chlorine-e₆, or a synthetic polymer, or a biopolymer; and
d) a conjugate of the compound characterized above in sub-items a) or b), with an amino compound;
and a pharmaceutically acceptable excipient or solvent.

2. The composition of claim 1, in which the amino acid, covalently bonded to the fullerene C₆₀ molecule is an L- or D-amino acid selected from the group consisting of (international three-letter code): Ala, Arg, Asn, Asp, Cys, Gly, Glu, Gin, His, Ile, Leu, Lys, Met, Phe, Pro, Ser, Thr, Trp, ??r, Val, γ-aminobutyric acid (Aba), ε-aminocaproic acid (Acp), citrulline, ornithine.

3. The composition of claim 1, in which the dipeptide covalently bonded to the fullerene C₆₀ is a dipeptide comprising two L- or D-amino acids independently selected from the group consisting of (international three-letter code): Ala, Arg, Asn, Asp, Cys, Gly, Glu, Gln, His, Ile, Leu, Lys, Met, Phe, Pro, Ser, Thr, Trp, Tyr, Val, γ-aminobutyric acid (Aba), ε-aminocaproic acid (Acp), citrulline, ornithine.

4. A method of photodynamic therapy of a human oncological disease, characterized in that a patient in need of such treatment is administered as a photosensitizer the pharmaceutical composition according to claims 1.

5. The method of claim 4, in which the oncological disease is selected from the group consisting of carcinomas, sarcomas, lymphomas, leukaemias, blastomas.

6. The method of claim 4, in which the pharmaceutical composition is administered intramuscularly, intraperitoneally, topically, orally, intravenously, intratumorally, intranasally.

7. The method of claim 4, in which the pharmaceutical composition is administered in a daily dose amounting to from 0.5 to 50 mg/kg body weight.

8. A method of photodynamic therapy of a human oncological disease, characterized in that a patient in need of such treatment is administered as a photosensitizer the pharmaceutical composition according to claim 2.

9. A method of photodynamic therapy of a human oncological disease, characterized in that a patient in need of such treatment is administered as a photosensitizer the pharmaceutical composition according to claim 3.
